(19) United States
(12) Patent Application Publication
(10) Pub. No.: US 2018/0338544 A1
(43) Pub. Date: Nov. 29, 2018

(54) FABRIC MODULE AND SMART FABRIC USING THE SAME

(71) Applicant: TAIWAN TEXTILE RESEARCH INSTITUTE, NEW TAIPEI CITY (TW)

(72) Inventors: Tzu-Hao HUANG, NEW TAIPEI CITY (TW); Chien-Lung SHEN, NEW TAIPEI CITY (TW); Po-Chun HSU, NEW TAIPEI CITY (TW); Hou-Sheng HUANG, NEW TAIPEI CITY (TW); Wen-Kai TU, NEW TAIPEI CITY (TW); Chien-Fa TANG, NEW TAIPEI CITY (TW)

(21) Appl. No.: 15/852,069

(22) Filed: Dec. 22, 2017

(30) Foreign Application Priority Data
May 26, 2017 (TW) 106117728
May 26, 2017 (TW) 106207659

(51) Int. Cl. A41D 1/00; H05K 1/02; H05K 1/03; H05K 1/14; H05K 1/09; H05K 1/18; H05K 3/28; H05K 3/30; H05K 3/36; H02J 7/02; A41B 1/08; A61B 5/04; A61B 5/0408; A61B 5/0492; A61B 5/00

(52) U.S. Cl. CPC A41D 1/005; H01L 25/0753; H05K 1/038; H05K 1/144; H05K 1/09; H05K 1/189; H05K 3/285; H05K 3/303; H05K 3/361; H02J 7/025; A41B 1/08; A61B 5/04001; A61B 5/04085; A61B 5/0492; A61B 5/6804; H05K 2201/0129; H05K 1/0283

(57) ABSTRACT

A fabric module includes a first textile, a first elastic waterproof film, a second elastic waterproof film, a first conductive pattern, a control module, and a second textile. The first elastic waterproof film is disposed on the first textile. The second elastic waterproof film is disposed on the first elastic waterproof film. The first conductive pattern is enclosed between the first and second elastic waterproof films and adheres to a surface of one of the first and second elastic waterproof films. The control module is disposed on the first textile and electrically connected to the first conductive pattern. The second textile is opposite to the first textile, in which the first elastic waterproof film, the second elastic waterproof film, the first conductive pattern, and the control module are present between the first and second textiles.

### RELATED APPLICATIONS

This application claims priority to Taiwanese Application Serial Number 106207659, filed May 26, 2017, and to Taiwanese Application Serial Number 106117728, filed May 26, 2017. The entire disclosure of the above application is hereby incorporated by reference herein.

### BACKGROUND

### Technical Field

The present disclosure relates to a fabric module and a smart fabric using the same.

### Description of Related Art

In recent years, with the development of wearable devices, many electronic devices have been designed in a wearable type, such as smart watches, wearable pedometers, smart bracelets, or the like. Moreover, with the prevalence of smart products nowadays, these wearable electronic devices have also become mainstream items in the consumer market. On the other hand, since these wearable electronic devices have had a great response in the consumer market, the combination of electronic devices and apparel has been launched into the consumer market one after another. Furthermore, e-commerce has also started to be in alliance with traditional textiles, such that the development of functional electronic products using fabrics are attracted attention.

### SUMMARY

An aspect of the present disclosure provides a smart fabric including two textiles and a fabric module, in which the fabric module includes more than two elastic waterproof films, at least one conductive pattern, and a control module. The conductive pattern and the control module are enclosed between the elastic waterproof films, and the elastic waterproof films are disposed between the two textiles. According to such configuration, the conductive pattern and the control module can be enclosed in the space between the elastic waterproof films, so as to avoid affecting by moisture or dust. Accordingly, the smart fabric is washable.

An aspect of the present disclosure provides a fabric module including a first textile, a first elastic waterproof film, a second elastic waterproof film, a first conductive pattern, a control module, and a second textile. The first elastic waterproof film is disposed on the first textile. The second elastic waterproof film is disposed on the first elastic waterproof film. The first conductive pattern is enclosed between the first and second elastic waterproof films and adheres to a surface of the first elastic waterproof film or the second elastic waterproof film. The control module is disposed on the first textile and electrically connected to the first conductive pattern. The second textile is opposite to the first textile, in which the first elastic waterproof film, the second elastic waterproof film, and the control module are present between the first and second textiles.

In some embodiments, the control module includes a controller and a flexible circuit board. The controller is disposed between the first and second elastic waterproof films. The flexible circuit board is disposed between the first and second elastic waterproof films, in which the controller is electrically connected to the first conductive pattern through the flexible circuit board.

In some embodiments, the control module includes a controller and an anisotropic conductive film. The controller is disposed between the first and second elastic waterproof films. The controller is electrically connected to the first conductive pattern through the anisotropic conductive film.

In some embodiments, the first conductive pattern adheres to the surface of the first elastic waterproof film, and the fabric module further includes a third elastic waterproof film and a second conductive pattern. The third elastic waterproof film is disposed between the second elastic waterproof film and the second textile. The second conductive pattern adheres to a surface of the second elastic waterproof film and is enclosed between the second and third elastic waterproof films, in which the control module is electrically connected to the second conductive pattern.

In some embodiments, the first conductive pattern has a plurality of first row patterns extending along a first direction, and the second conductive pattern has a plurality of second row patterns extending along a second direction which intersects the first direction.

In some embodiments, the control module comprises a controller and a flexible circuit board. The controller is disposed between the first and third elastic waterproof films. The flexible circuit board is disposed between the first and third elastic waterproof films, in which the controller is electrically connected to the first and second conductive patterns through the flexible circuit board.

In some embodiments, the control module includes a controller and an anisotropic conductive film. The controller is disposed between the first and third elastic waterproof films and has a plurality of pins. A vertical projection of the pins on the first elastic waterproof film partially overlaps with the first conductive pattern, and the vertical projection of the pins on the second elastic waterproof film partially overlaps with the second conductive pattern. The anisotropic conductive film is disposed at the pins of the controller, in which the controller is electrically connected to the first and second conductive patterns through the anisotropic conductive film.

In some embodiments, the first conductive pattern has a plurality of first row patterns extending along a first direction, and the fabric module further includes a second conductive pattern. The second conductive pattern is enclosed between the first and second elastic waterproof films, in which the first and second conductive patterns together adhere to the surface the first elastic waterproof film or the second elastic waterproof film. The second conductive pattern has a plurality of second row patterns extending along a second direction which intersects the first direction, and the first and second conductive patterns on the first elastic waterproof film partially overlap with each other.

In some embodiments, the second conductive patterns are made of an anisotropic conductive film, and the anisotropic conductive film is conductive in a third direction which intersects a plane composed of the first and second directions.

In some embodiments, the fabric module further comprises an electronic component. The electronic component is enclosed between the first and second elastic waterproof films and has a first pin and a second pin, in which the first and second pins are respectively located at overlapping regions of the first and second conductive patterns.

In some embodiments, the first conductive pattern includes a first conductive area and a second conductive area which are separated from each other. A portion of the anisotropic conductive film is located between the first pin and the first conductive area, and another portion of the anisotropic conductive film is located between the second pin and the second conductive area.

In some embodiments, the first and second elastic waterproof films comprise a thermoplastic urethane (TPU) material.

In some embodiments, the first conductive pattern comprises silver particles.

An aspect of the present disclosure provides a smart fabric including a first textile, a fabric module, and a second textile. The first textile has an inner surface and an outer surface. The fabric module is disposed at the inner surface of the first textile, in which the fabric module includes a first elastic waterproof film, a second elastic waterproof film, a first conductive pattern, and a control module. The first elastic waterproof film is disposed on the first textile. The second elastic waterproof film is disposed on the first elastic waterproof film. The first conductive pattern is enclosed between the first and second elastic waterproof films and adheres to a surface the first elastic waterproof film or the second elastic waterproof film. The control module is disposed on the first textile and electrically connected to the first conductive pattern. The second textile is opposite to the first textile, in which the first elastic waterproof film, the second elastic waterproof film, and the control module are present between the first and second textiles.

In some embodiments, the first conductive pattern includes at least one detection electrode and a conductive path, and a thickness of each of the detection electrode and the conductive path is in a range from 10 μm to 20 μm.

In some embodiments, the first conductive pattern adheres to the first elastic waterproof film. The second elastic waterproof film and the second textile collectively have an opening, and the first conductive pattern is exposed from the opening.

In some embodiments, the fabric module is a detection module, and the first conductive pattern exposed from the opening is a detection electrode.

In some embodiments, the control module is enclosed between the first and second elastic waterproof films, and the control module includes a wireless charger and a wireless emitting-and-receiving device.

### BRIEF DESCRIPTION OF THE DRAWINGS

FIGS. 1A and 1B are front views of a smart fabric according to a first embodiment of the present disclosure;

FIG. 1C is an exploded view of a configuration within the area C in FIG. 1B;

FIG. 1D is an enlarged drawing of the configuration within the area C in FIG. 1B;

FIG. 1E is a graph plotting an elongation ratio versus tension force in a tension test performed to a smart fabric;

FIG. 1F is a graph plotting an elongation ratio versus a change of resistance in a tension test performed to a smart fabric;

FIG. 2 is a partial enlarged drawing of a smart fabric according to a second embodiment of the present disclosure;

FIG. 3A is an exploded view of a fabric module according to a third embodiment of the present disclosure;

FIG. 3B is a top view of the first elastic waterproof film and a first conductive pattern thereon of the fabric module illustrated in FIG. 3A;

FIG. 3C is a top view of the second elastic waterproof film and a second conductive pattern thereon of the fabric module illustrated in FIG. 3A;

FIG. 3D is a top view of the fabric module illustrated in FIG. 3A;

FIG. 3E is a flowchart of a method for forming the fabric module illustrated in FIG. 3A;

FIG. 3F is a graph plotting an elongation ratio versus tension force in a tension test performed to a fabric module;

FIG. 3G is a graph plotting an elongation ratio versus a change of capacitance in a tension test performed to a fabric module;

FIG. 4 is a top view of a smart textile according to a fourth embodiment of the present disclosure;

FIG. 5A is an exploded view of a smart textile according to a fifth embodiment of the present disclosure;

FIG. 5B is a top view of the first and second elastic waterproof films and the second conductive pattern of the fabric module illustrated in FIG. 5A;

FIG. 5C is a configuration of electronic components of the fabric module;

FIG. 5D is a flowchart of a method for forming the fabric module illustrated in FIG. 5A; and

FIG. 6 is a top view of a smart textile according to a sixth embodiment of the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

It will be understood that, although the terms first, second, third etc. may be used herein to describe various elements, components, regions, layers and/or sections, these elements, components, regions, layers and/or sections should not be limited by these terms.

In the following detailed description, the term “electrical connection” or the like can be achieved by a wireless connection or a wired connection. As the electrical connection is achieved by a wireless connection, the wireless may be realized by a Bluetooth transmission device, an infrared transmission device, a WIFI wireless network transmission device, a WT radio transmission device, an NFC short distance wireless communication device, an ANT+ short distance wireless communication device, or a Zigbee communication device. As the electrical connection is achieved by a wired connection, the wired connection may be realized by a physical cable, in which the physical cable may include a high definition multimedia interface (HDMI), a controller area network area network; CANbus), RS-232 or Ethernet Control Automation Technology (etherCAT).

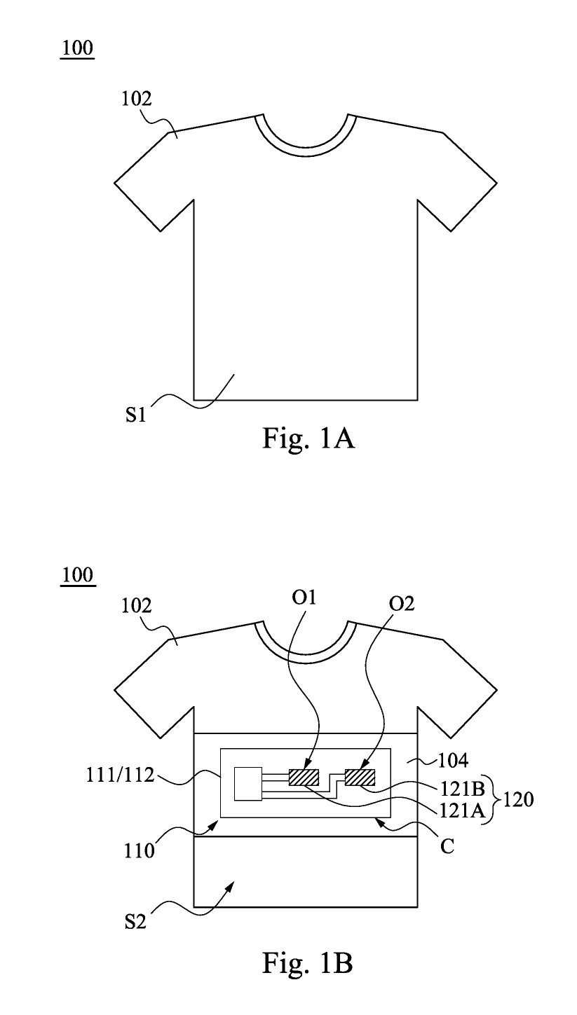

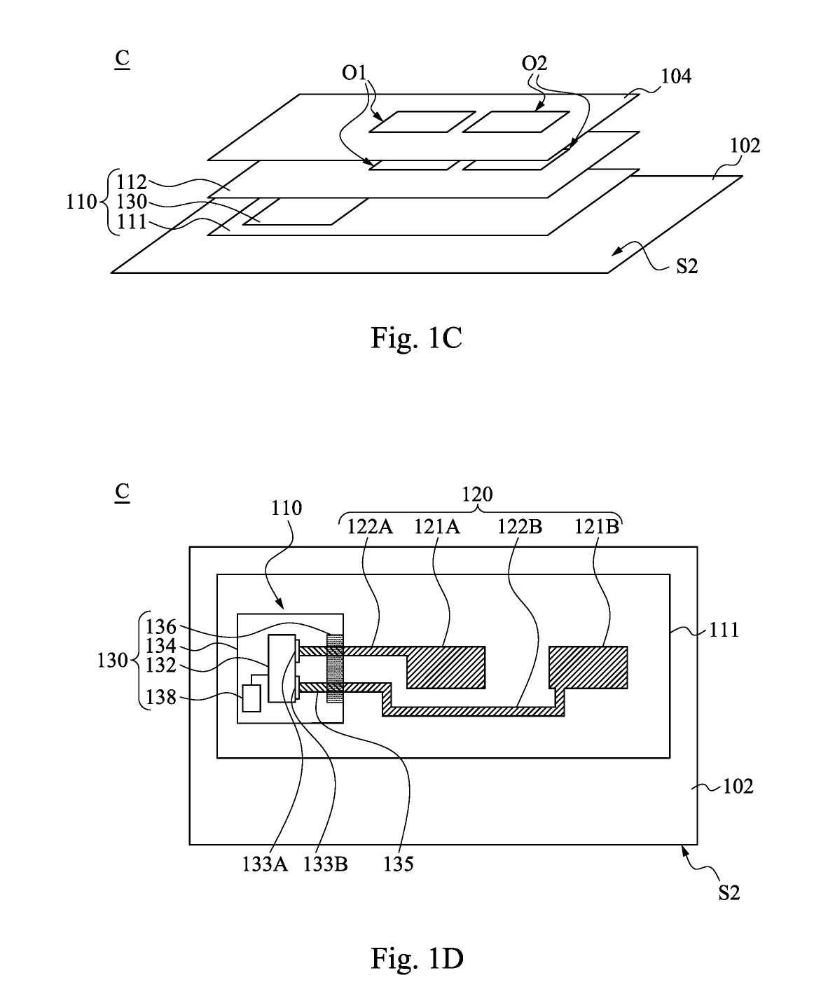

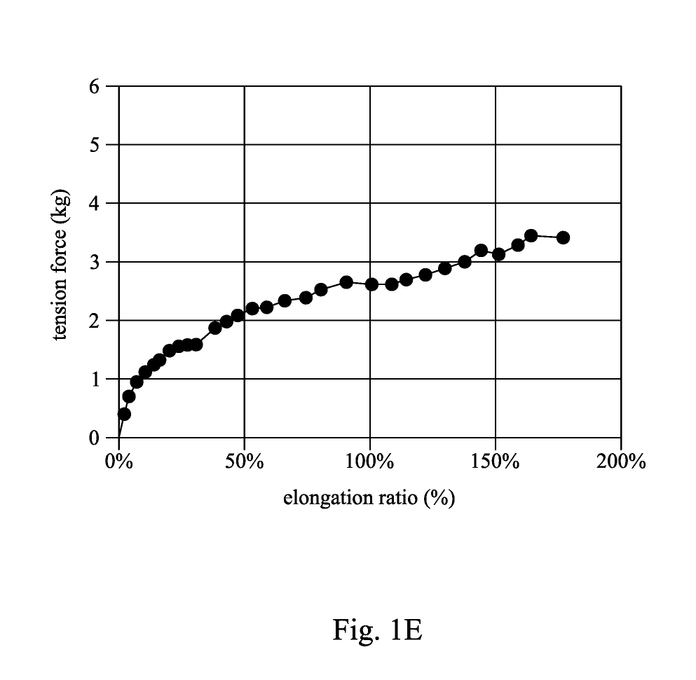

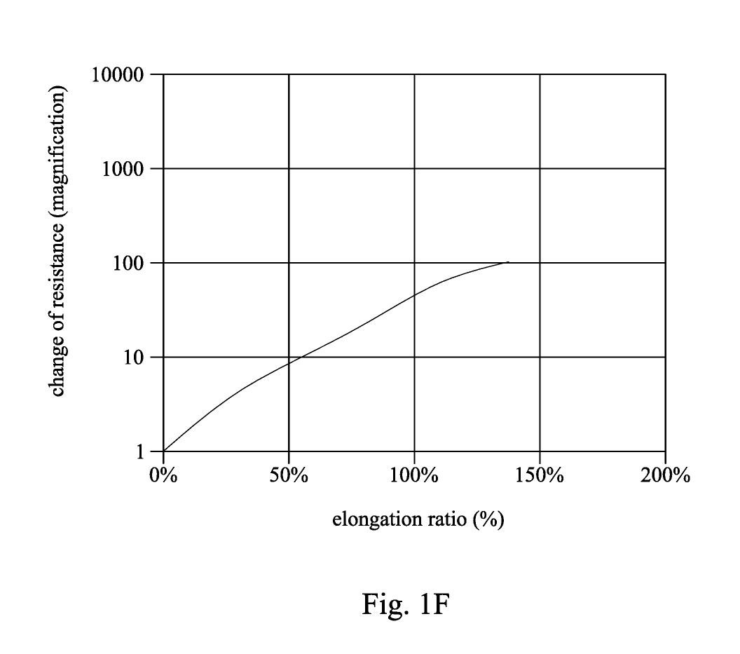

FIGS. 1A and 1B are front views of a smart fabric 100 according to a first embodiment of the present disclosure, in which the smart fabric 100 illustrated in FIG. 1B shows turning an inner of the smart fabric 100 of FIG. 1A outside. As shown in FIGS. 1A and 1B, the smart fabric 100 includes a first textile 102, a second textile 104, and a fabric module 110. The first textile 102 can serve as a textile body of the smart fabric 100. In the present embodiment, although the appearance of the smart fabric 100 is illustrated as a t-shirt, the smart fabric 100 can be designed in other types. For example, in other embodiments, the smart fabric 100 may be a sportswear, a sportswear, a heartbeat webbing, a leg cuff, a wristband, a glove, or sweatpants.

The first textile 102 has an outer surface S1 and an inner surface S2 which are respectively shown in FIGS. 1A and 1B. The second textile 104 and the fabric module 110 are disposed on the inner surface S2 of the first textile 102, and the fabric module 110 is partially covered with the second textile 104. The fabric module 110 may include a control module and at least one conductive pattern electrically connected to the control module, so as to make the smart fabric 100 functional through the fabric module 110. The following descriptions are provided with respect to such functionality.

FIG. 1C is an exploded view of a configuration within the area C in FIG. 1B, and FIG. 1D is an enlarged drawing of the configuration within the area C in FIG. 1B. As shown in FIG. 1C, the fabric module 110 includes a first elastic waterproof film 111, a second elastic waterproof film 112, and a control module 130, in which the first and second elastic waterproof films 111 and 112, the first conductive pattern 120, and the control module 130 are present between the first and second textiles 102 and 104.

The first elastic waterproof film 111 is disposed on the inner surface S2 of the first textile 102, and the second elastic waterproof film 112 is disposed on the first elastic waterproof film 111. The control module 130 is disposed between the first and second elastic waterproof films 111 and 112. The first and second elastic waterproof films 111 and 112 include a thermoplastic urethane (TPU) material therein. In addition, the second textile 104 and the second elastic waterproof film 112 may collectively have openings O1 and O2.

Next, as shown in FIG. 1D, the first conductive pattern 120 adheres to a surface of the first elastic waterproof film 111, and the first conductive pattern 120 is present between the first and second elastic waterproof films 111 and 112. In addition, although the first conductive pattern 120 illustrated in FIG. 1D adheres to the surface of the first elastic waterproof film 111, the first conductive pattern 120 may adhere to a surface of the second second elastic waterproof film 112 in other embodiments and be present between the first and second elastic waterproof films 111 and 112. The first conductive pattern 120 may be formed by arranging conductive ink on the surface of the first elastic waterproof film 111. For example, the first conductive pattern 120 may be made of the conductive ink, and the conductive ink is a silver adhesive including silver particles therein.

The first conductive pattern 120 includes detection electrodes 121A and 121B and conductive paths 122A and 122B. The detection electrode 121A and the conductive path 122A are connected to each other, and the detection electrode 121B and the conductive path 122B are connected to each other. Furthermore, in embodiments that the first conductive pattern 120 is formed by the conductive ink, the detection electrodes 121A and 121B and the conductive paths 122A and 122B of the first conductive pattern 120 may have the same thickness in a range from 10 μm to 20 μm. That is, in order to form the first conductive pattern 120, the detection electrodes 121A and 121B and the conductive paths 122A and 122B can be formed by the same conductive ink, and therefore the thickness of each of the detection electrodes 121A and 121B and the conductive paths 122A and 122B may be in a range from 10 μm to 20 μm. On the other hand, the detection electrodes 121A and 121B of the first conductive pattern 120 can be exposed from the openings O1 and O2 collectively defined by the second textile 104 and the second elastic waterproof film 112.

The control module 130 includes a controller 132, a flexible circuit board 134, an anisotropic conductive film 136, and a wireless module 138. The controller 132, the flexible circuit board 134, the anisotropic conductive film 136, and the wireless module 138 are disposed between the first and second elastic waterproof films 111 and 112.

The controller 132 has pins 133A and 133B. The flexible circuit board 134 has at least one blind via (not illustrated) and a wire pattern 135. The wire pattern 135 can be in contact with the conductive paths 122A and 122B of the first conductive pattern 120. The pins 133A and 133B of the controller 132 can be electrically connected to the wire pattern 135 through the blind via of the flexible circuit board 134, such that the pins 133A and 133B of the controller 132 can respectively electrically connected to the conductive paths 122A and 122B of the first conductive pattern 120.

The anisotropic conductive film 136 has electrical conductibility in a normal direction of the surface of the first elastic waterproof film 111. That is, the anisotropic conductive film 136 is electrically conductive in a direction perpendicular to FIG. 1D. The anisotropic conductive film 136 is located between the first conductive pattern 120 and the wire pattern 135 and is in contact with the first conductive pattern 120 and the wire pattern 135, so as to enhance electrical reliability of the first conductive pattern 120 and the wire pattern 135. Furthermore, although the anisotropic conductive film 136 illustrated in FIG. 1D is strip-shaped and may be form by a solid anisotropic conductive tape, the anisotropic conductive film 136 may be dot-shaped and formed by dispensing a liquid anisotropic conductive film in other embodiments.

The wireless module 138 is electrically connected to the controller 132 and includes a wireless emitting-and-receiving device and a wireless charging device. By the wireless emitting-and-receiving device and the wireless charging device of the wireless module 138, even if the control module 130 is enclosed between the first and second elastic waterproof films 111 and 112, the control module 130 can communicate to an external device or can be charged.

In the present embodiment, when the smart fabric 100 shown in FIG. 1A or FIG. 1B is worn, a combination of the first conductive pattern 120 and the control module 130 can be configured to detect a physiological signal, such as an electrocardiography signal, an electromyogram signal, and an electroneurogram signal. More specifically, as shown in FIGS. 1B and 1C, when the smart fabric 100 is worn, the detection electrodes 121A and 121B can be exposed from the openings O1 and O2 collectively defined by the second textile 104 and the second elastic waterproof film 112, so as to contact with skin. In such case, the exposed detection electrodes 121A and 121B can correspondingly server as a pair of electrocardiography electrodes, electromyogram electrodes, or electroneurogram electrodes.

On the other hand, as the conductive pattern is formed by using the conductive ink, the conductive pattern can adhere to the surface of the elastic waterproof film. Therefore, when the first textile or the elastic waterproof film is tensed, the conductive pattern may not be damaged easily such that the conductive pattern can still detect the physiological signal under tension. The following descriptions are provided with respect to a tension test performed on the smart fabric. In the tension test, a tension force is applied to the smart fabric, and an elongation ratio and a change of resistance are measured during applying the tension force.

FIG. 1E is a graph plotting an elongation ratio versus tension force in a stretching test performed to a smart fabric. In FIG. 1E, the horizontal axis represents an elongation ratio of the smart fabric as the unit percentage, and the vertical axis represents the tension force applying to the smart fabric as the unit kilogram. FIG. 1F is a graph plotting an elongation ratio versus a change of resistance in a stretching test performed to a smart fabric. In FIG. 1E, the horizontal axis represents an elongation ratio of the smart fabric as the unit percentage, and the vertical axis represents the change of the resistance as the unit magnification.

As collectively shown in FIGS. 1E and 1F, as the smart fabric is tensed and the elongation ratio thereof is less than 150%, the resistance of the smart fabric is still stable. That is, even though the smart fabric is deformed due to the tension, the conductive pattern thereof would not become open circuit by the damage caused from the deformation. Accordingly, the smart fabric is stretchable.

According to the above, the smart fabric of the present embodiment includes the first textile, the second textile, and the fabric module, in which the fabric module includes the two elastic waterproof films, the conductive pattern, and the control module. The fabric module can be provided a function for detecting a physiological signal through the conductive pattern and the control module. The conductive pattern and the control module are enclosed between the two elastic waterproof films, and therefore the electrical properties of the conductive pattern and the control module are protected from moisture, so as to make the smart fabric washable. On the other hand, when the smart fabric is tensed, the conductive pattern thereon would not become open circuit by the deformation, so as to make the smart fabric stretchable.

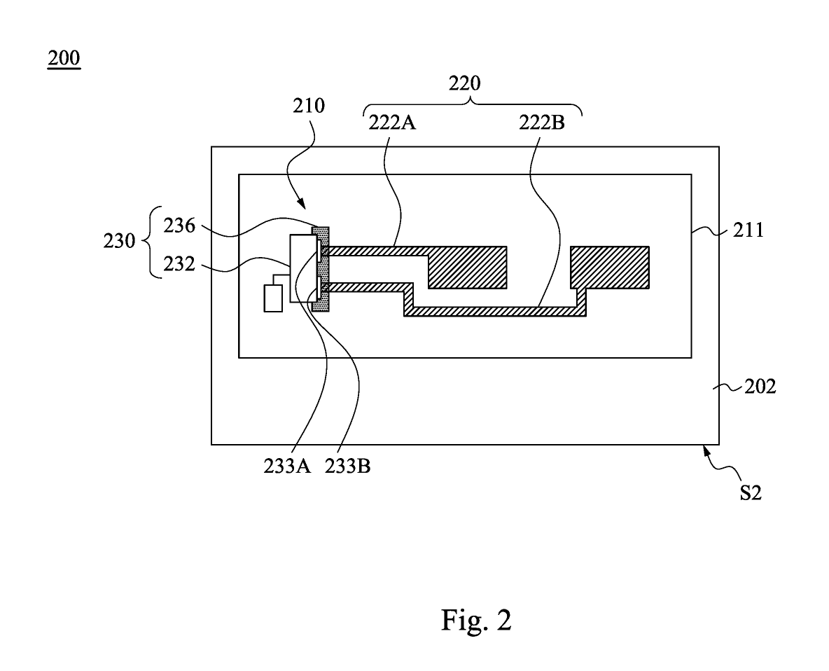

FIG. 2 is a partial enlarged drawing of a smart fabric 200 according to a second embodiment of the present disclosure. The smart fabric 200 of the present embodiment includes a first textile 202, a second textile (not illustrated in FIG. 2), a first elastic waterproof film 211, a second elastic waterproof film (not illustrated in FIG. 2), a first conductive pattern 220, and a control module 230, and the smart fabric 200 has a configuration which is similar to that of the smart fabric 100 of the first embodiment. At least one difference between the smart fabric 200 of the present embodiment and the smart fabric 100 of the first embodiment is that the flexible circuit board (e.g., the flexible circuit board 134) is replaced by an anisotropic conductive film 236 in the control module 230. Accordingly, the control module 230 may include a controller 232 having pins 233A and 233B, and the pins 233A and 233B of the controller 232 are fixed to conductive paths 222A and 222B of the first conductive pattern 220 by the anisotropic conductive film 236. The anisotropic conductive film 236 is only conductive in a direction which can referred to as a normal direction of the FIG. 2. Therefore, the pins 233A and 233B of the controller 232 are electrically connected to the first conductive pattern 220 through the anisotropic conductive film 236.

According to the above embodiments, the smart fabric can be provided a function of detecting a physiological signal. Besides the detecting the physiological signal, the smart fabric can be provided other function, such as a touch function or a light-emitting function, by difference configuration of the fabric module. The following descriptions are provided with respect to the other functions.

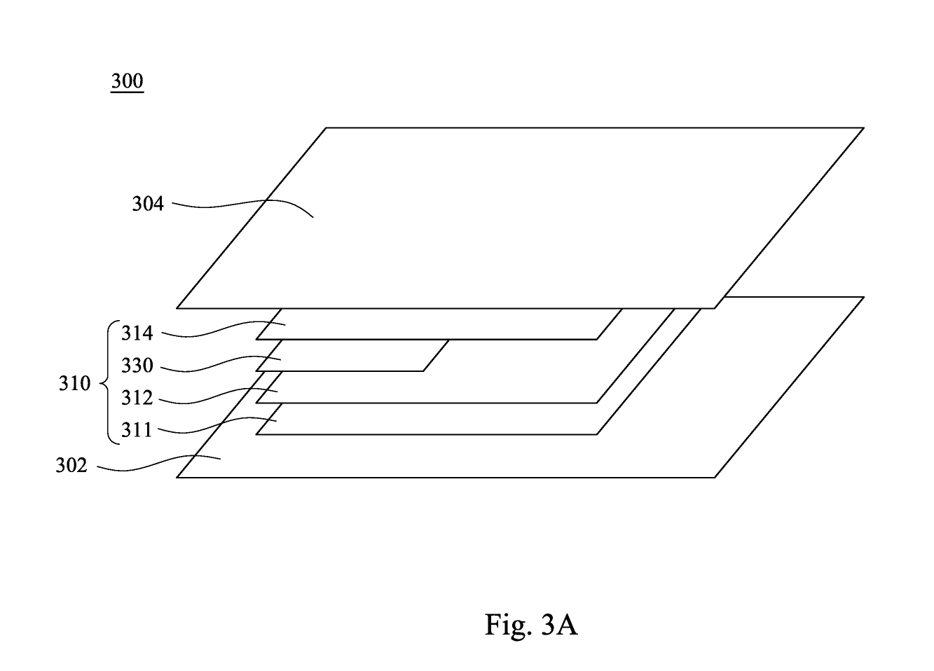

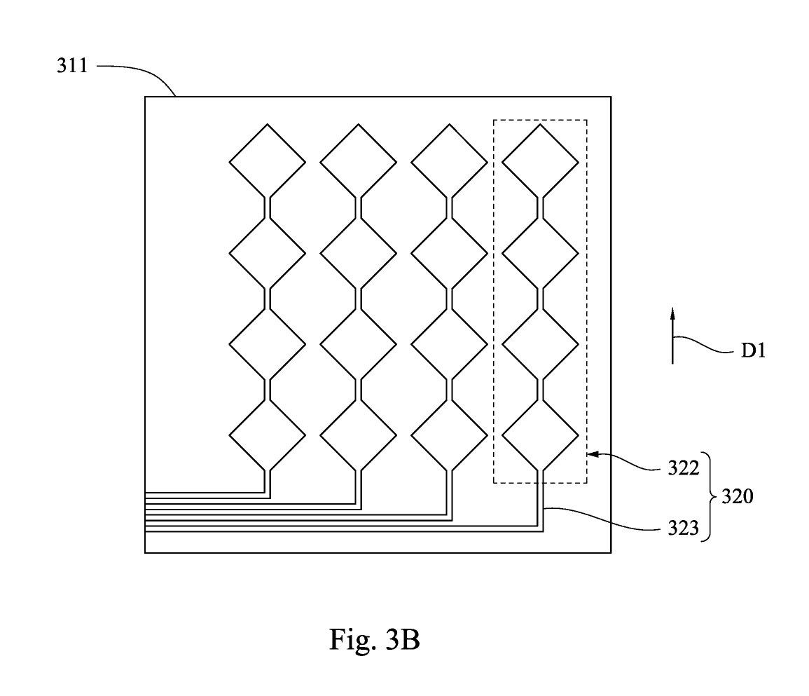

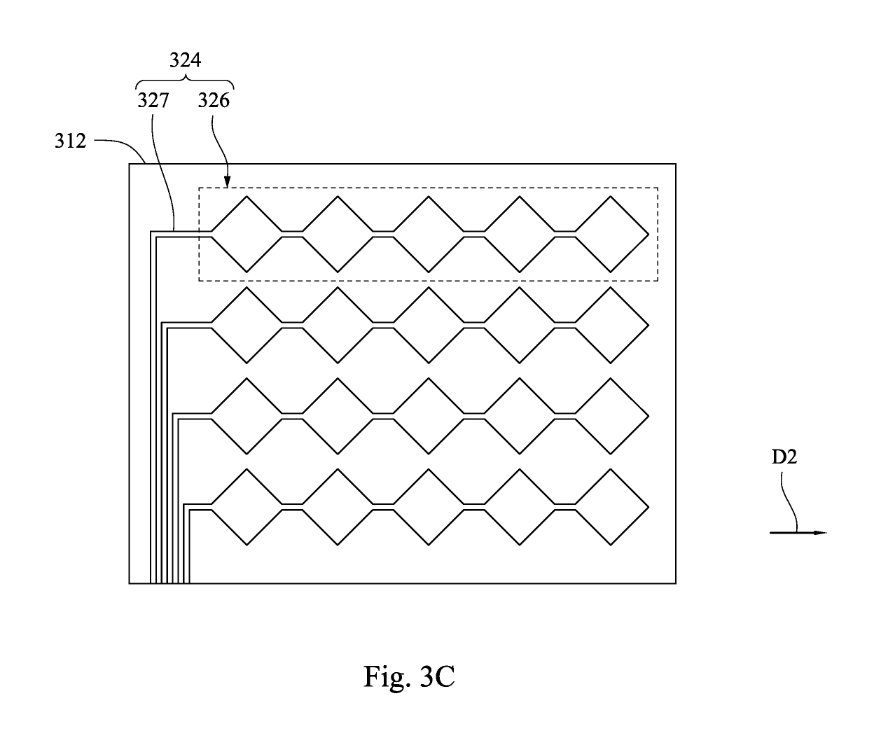

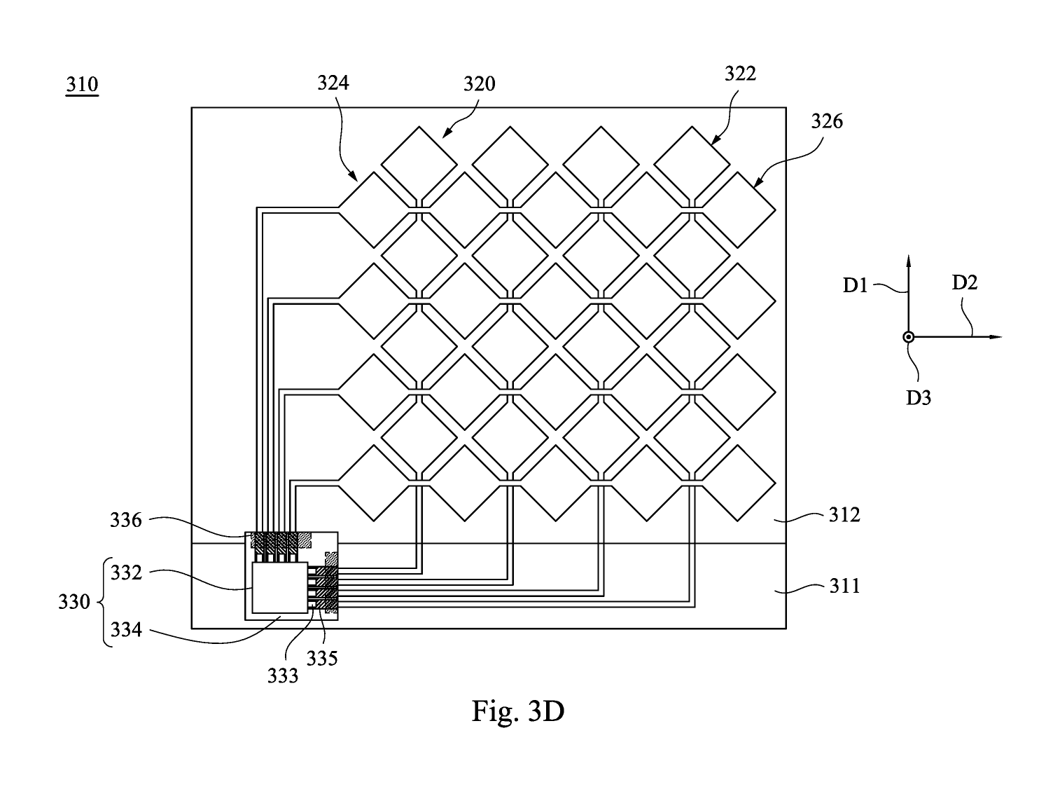

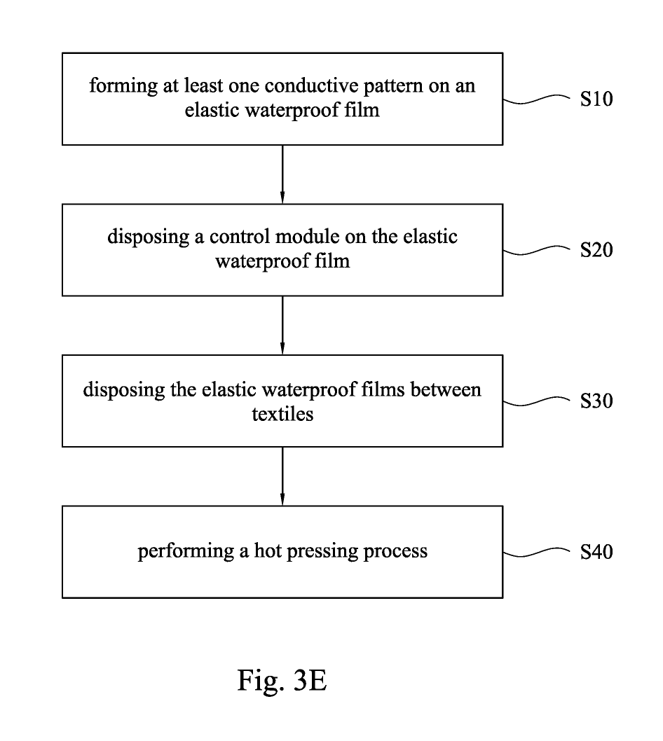

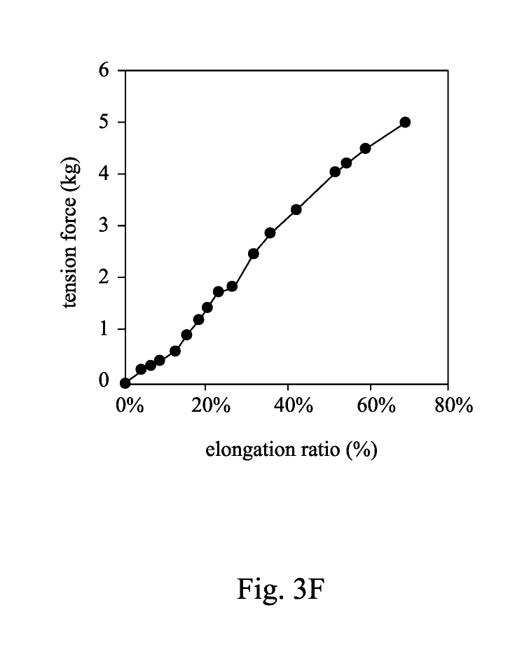

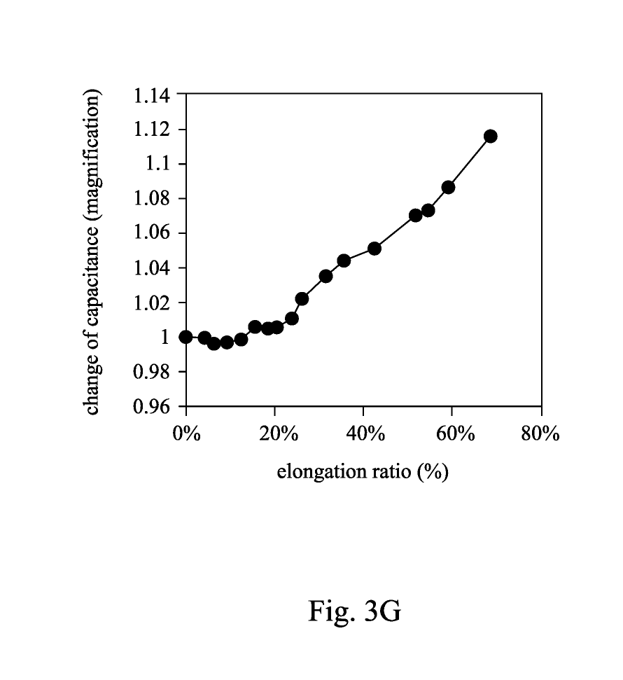

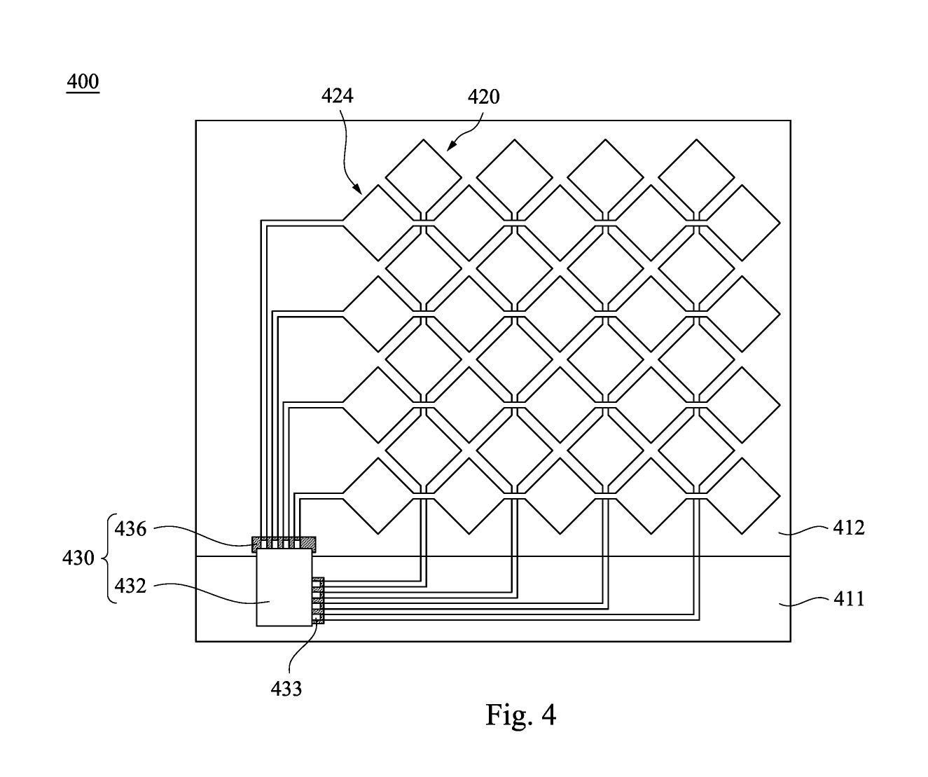

FIG. 3A is an exploded view of a smart fabric 300 according to a third embodiment of the present disclosure. The smart fabric 300 of the present embodiment includes a first textile 302, a second textile 304, and a fabric module 310, in which the first textile 302 and the second textile 304 are opposite to each other. For making the description succinct, the first textile 302 and the second textile 304 are only partially illustrated in FIG. 3A.

The fabric module 310 includes a first elastic waterproof film 311, a second elastic waterproof film 312, a third elastic waterproof film 314, and a control module 330, which are all enclosed between the first and second textiles 302 and 304.

The first elastic waterproof film 311, the second elastic waterproof film 312, and the third elastic waterproof film 314 are arranged by stacking. The first elastic waterproof film 311 is disposed on the first textile 302, the second elastic waterproof film 312 is disposed on the first elastic waterproof film 311, and the third elastic waterproof film 314 is disposed on the second elastic waterproof film 312. In some embodiments, the first, second, and third elastic waterproof films may comprise a TPU material.

The control module 330 is disposed between the first and second elastic waterproof films 311 and 312, but is not limited thereto. For example, in other embodiments, the control module 330 may be located at other position between the first and second textile 302 and 304. In addition, the fabric module 310 further includes at least one conductive pattern, and the conductive pattern can be coupled to the elastic waterproof film and electrically connected to the control module 330. By a combination of the conductive pattern and the control module 330, the smart fabric 300 can be provided a touch function as described below.

FIG. 3B is a top view of the first elastic waterproof film 311 and a first conductive pattern 320 thereon of the fabric module 310 illustrated in FIG. 3A, in which the “top view” means FIG. 3B is viewed from the second elastic waterproof film 312 to the first elastic waterproof film 311 of FIG. 3A. As shown in FIG. 3B, the first conductive pattern 320 adheres to a surface of the first elastic waterproof film 311, and the first conductive pattern 320 has a plurality of first row patterns 322 and a plurality of first conductive-path patterns 323. The first row patterns 322 extend along a first direction D1, and the first row patterns 322 are electrically isolated from each other. The first conductive-path patterns 323 respectively extend from ends of the first row patterns 322 to an edge of the surface of the first elastic waterproof film 311. The first conductive pattern 320 may include conductive particles therein. For example, the first conductive pattern 320 is a silver adhesive including silver particles therein.

FIG. 3C is a top view of the second elastic waterproof film 312 and a second conductive pattern 324 thereon of the fabric module 310 illustrated in FIG. 3A, in which FIG. 3C is a view from the third elastic waterproof film 314 to the second elastic waterproof film 312 of FIG. 3A. As shown in FIG. 3C, the second conductive pattern 324 adheres to a surface of the second elastic waterproof film 312, in which the second conductive pattern 324 and the first conductive pattern 320 illustrated in FIG. 3B can be separated from each other by the second elastic waterproof film 312. The second conductive pattern 324 has a plurality of second row patterns 326 and a plurality of second conductive-path patterns 327. The second row patterns 326 extend along a second direction D2, and the second row patterns 326 are electrically isolated from each other. The second direction D2 can intersect the first direction D1. For example, the second direction D2 may be orthogonal to the first direction D1. The second conductive-path patterns 327 respectively extend from ends of the second row patterns 326 to an edge of the surface of the second elastic waterproof film 312. The second conductive pattern 320 may have the same material as the first conductive pattern 320. For example, the second conductive pattern 320 may be a silver adhesive including silver particles therein.

FIG. 3D is a top view of the fabric module 310 illustrated in FIG. 3A, and the first textile 302, the second textile 304, and the third elastic waterproof film 314 illustrated in FIG. 3A are omitted in FIG. 3D. As shown in FIG. 3D, the control module 330 includes a controller 332, a flexible circuit board 334, and an anisotropic conductive film 336, in which the controller 332, the flexible circuit board 334, and the anisotropic conductive film 336 are disposed on the first and second elastic waterproof films 311 and 312.

The controller 332 has a plurality of pins 333. The flexible circuit board 334 has at least one blind via (not illustrated in FIG. 3D) and a wire pattern 335, in which the pins 333 of the controller 332 can be electrically connected to the wire pattern 335 through the blind via. The wire pattern 335 can be in contact with the first conductive pattern 320 on the first elastic waterproof film 311 and the second conductive pattern 324 on the second elastic waterproof film 312.

The anisotropic conductive film 336 may be conductive only in a third direction D3, in which the third direction D3 can intersect a plane composed of the first and second directions D1 and D2. For example, the third direction D3 can be referred to as a normal direction of the FIG. 3D. The anisotropic conductive film 336 disposed on the first elastic waterproof film 311 may be disposed between the first conductive pattern 320 and the wire pattern 335 and be contacted with the first conductive pattern 320 and the wire pattern 335, thereby enhancing electrical reliability between the first conductive pattern 320 and the wire pattern 335. Similarly, the anisotropic conductive film 336 disposed on the second elastic waterproof film 312 may be disposed between the second conductive pattern 324 and the wire pattern 335 and be contacted with the second conductive pattern 324 and the wire pattern 335, thereby enhancing electrical reliability therebetween. By the wire pattern 335 of the flexible circuit board 334 and the anisotropic conductive film 336, the pins 333 of the controller 332 can be electrically connected to the row patterns of the conductive patterns.

According to the above configuration, the first row patterns 322 of the first conductive pattern 320 and the second row patterns 326 of the second conductive pattern 324 can serve as touch electrodes. For example, the first row patterns 322 of the first conductive pattern 320 can serve as transmit (TX) electrodes, and the second row patterns 326 of the second conductive pattern 324 can serve as receive (RX) electrodes. The controller 332 can take coupling capacitance produced between the TX electrodes and the RX electrodes as a detection basis regarding the touching, and therefore the fabric module 300 can be provided a touch function.

Reference is made back to FIG. 3A. The conductive patterns or the electronic component can be enclosed between the elastic waterproof films and protected from outer environment, such as moisture or dust. In this regard, the first conductive pattern 320 of FIG. 3B can be enclosed between the first and second elastic waterproof films 311 and 312, and the second conductive pattern 324 of FIG. 3C can be enclosed between the second and third elastic waterproof films 312 and 314. Furthermore, the control module 330 illustrated in FIG. 1D can be disposed between the first and third elastic waterproof films 311 and 314, and the control module 330 can include a wireless emitting-and-receiving device and a wireless charging device, such that the control module 330 can be operated in the space between the elastic waterproof films. With such configuration, since the conductive patterns and the electronic component can be enclosed between the elastic waterproof films, the conductive patterns and the electronic components can be protected if the fabric module 300 was putted into liquid, such as water, so that the fabric module 300 can be washable. Moreover, the adjacent elastic waterproof films can adhere to each other, and the first and third elastic waterproof films 311 and 314 can respectively adhere to the first textile 302 and the second textile 304, so that the space between the elastic waterproof films can be sealed. The stickiness of the elastic waterproof films can be induced by a hot pressing process.

For example, FIG. 3E is a flowchart of a method for forming the fabric module 300 illustrated in FIG. 3A. As shown in FIG. 3E, the method for forming the fabric module 300 includes operations S10-S40.

The operation S10 is performed by forming at least one conductive pattern on an elastic waterproof film. In the operation S10, the first and second conductive patterns can be respectively formed by applying at least one conductive ink to the first and second elastic waterproof films. The conductive ink may include a silver adhesive. Then, a baking process can be performed on the elastic waterproof film with the silver adhesive thereon, such that the silver adhesive can adhere to the surface of the elastic waterproof film, thereby improving reliability of the conductive pattern. In some embodiments, a temperature in the baking process may be about 100° C., and a time thereof may be about ten minutes.

The operation S20 is performed by disposing a control module on the elastic waterproof film. In the operation S20, the controller can be bonded to the flexible circuit board. Then, the second elastic waterproof film is disposed on the first elastic waterproof film, and the first conductive pattern is covered with the second elastic waterproof film. Next, the anisotropic conductive film can be arranged on the first and second conductive patterns, and the anisotropic conductive film can be heated to about 90° C., so as to enhance adhesive strength of the anisotropic conductive film. Furthermore, although the anisotropic conductive film illustrated FIG. 3D is strip-shaped and may be a solid anisotropic conductive tape, the anisotropic conductive film may be dot-shaped and formed by dispensing a liquid anisotropic conductive film in other embodiments. After arranging the anisotropic conductive film, the controller and the flexible circuit board are disposed on the elastic waterproof film, in which the wire pattern of the flexible circuit board are aligned to and connected to the anisotropic conductive film. In addition, connecting the wire pattern of the flexible circuit board to the anisotropic conductive film can be performed under room temperature. After disposing the flexible circuit board, a hot pressing may be performed on the flexible circuit board and the anisotropic conductive film, so as to fix the flexible circuit board on the elastic waterproof film through the anisotropic conductive film. For example, the hot pressing in the operation S20 can be performed under a temperature of about 140° C. and a pressure of about 2 MPa.

The operation S30 is performed by disposing the elastic waterproof films between textiles. In the operation S30, the third elastic waterproof film can be arranged to cover the first and second elastic waterproof films, in which the control module is covered with the third elastic waterproof film as well. Then, the first and second textiles can be used for enclosing the first, second, and third elastic waterproof films and the control module disposed therebetween.

The operation S40 is performed by performing a hot pressing process. In the operation S40, the elastic waterproof film can be adhered to each other through the hot pressing process, and the first and third elastic waterproof films can be respectively adhered to the first and second textiles as well. For example, the hot pressing in the operation S40 can be performed under a temperature of about 140° C. and a pressure of about 2 MPa. After performing the hot pressing process on the elastic waterproof films, a manufacture process for the fabric module is finished.

On the other hand, as the conductive pattern is formed from the conductive ink, the conductive ink can be adhered to the surface of the elastic waterproof film. Therefore, when the elastic waterproof film is tensed, the conductive pattern thereon may not become an open circuit. Accordingly, the fabric module can still have a touch function.

The descriptions with respect to a tension test performed on the fabric module are provided in the following, so as to show an elongation ratio and a change of capacitance thereof during applying a tension force on the fabric module.

FIG. 3F is a graph plotting an elongation ratio versus tension force in a tension test performed to a fabric module. In FIG. 3F, the horizontal axis represents an elongation ratio of the fabric module as the unit percentage, and the vertical axis represents the tension force applying to the fabric module as the unit kilogram. FIG. 3G is a graph plotting an elongation ratio versus a change of capacitance in a tension test performed to a fabric module. In FIG. 3G, the horizontal axis represents an elongation ratio of the fabric module as the unit percentage, and the vertical axis represents the change of the capacitance as the unit magnification.

As shown in FIG. 3F, as the tension force applying to the fabric module increases to reach about 5 kg, the elongation ratio thereof gradually increases to reach about 80%, and no yield point occurs. Accordingly, as the elongation ratio of the fabric module is under 80%, no permanent deformation occurs. Then, as shown in FIG. 3G, as the elongation ratio of the fabric module gradually increases to reach about 80%, the change of the capacitance thereof gradually increases to reach about 1.12. Therefore, as collectively shown in FIGS. 3F and 3G, as the elastic waterproof film and the conductive pattern arranged thereon are tensed, the capacitance produced therefrom may not be varied greatly. That is, the conductive pattern arranged on the elastic waterproof film can be provided with the touch function under the elastic limit of the elastic waterproof film.

According to the above, the smart fabric of the present embodiment includes the first textile, the second textile, and the fabric module, in which the fabric module includes the elastic waterproof films, the conductive patterns, and the control module. The elastic waterproof films are enclosed between the first and second textiles. The conductive patterns and the control module are enclosed in the spaced between the elastic waterproof films, so as to avoid affecting by the moisture or the dust. Since the conductive patterns and the control module are enclosed in the space between the elastic waterproof films, the smart fabric is washable. On the other hand, the capacitance produced in the fabric module may not be varied greatly as the elastic waterproof film is tensed under the elastic limit thereof. Therefore, the fabric module is stretchable, and the touch function provided of the fabric module may not be affected when the fabric module is tensed.

FIG. 4 is a top view of a smart textile 400 according to a fourth embodiment of the present disclosure, in which the first textile, the second textile, and the third elastic waterproof film are omitted in FIG. 4. At least one difference between the smart fabric 400 of the present embodiment and the smart fabric 300 of the third embodiment is that the control module 430 of the fabric module 410 includes at least one anisotropic conductive film 436 to replace the flexible circuit board (e.g., the flexible circuit board 334), and thus the pins 433 of the controller 432 of the control module are directly fixed on the first and second conductive patterns 420 and 424 through the anisotropic conductive film 436.

Furthermore, at least one difference between a method for manufacturing the fabric module 400 and the method for manufacturing the fabric module 300 is that the controller 432 is directly disposed on the first and second elastic waterproof films 411 and 412, in which the pins 433 of the controller 432 are aligned to and connected to the anisotropic conductive film 436.

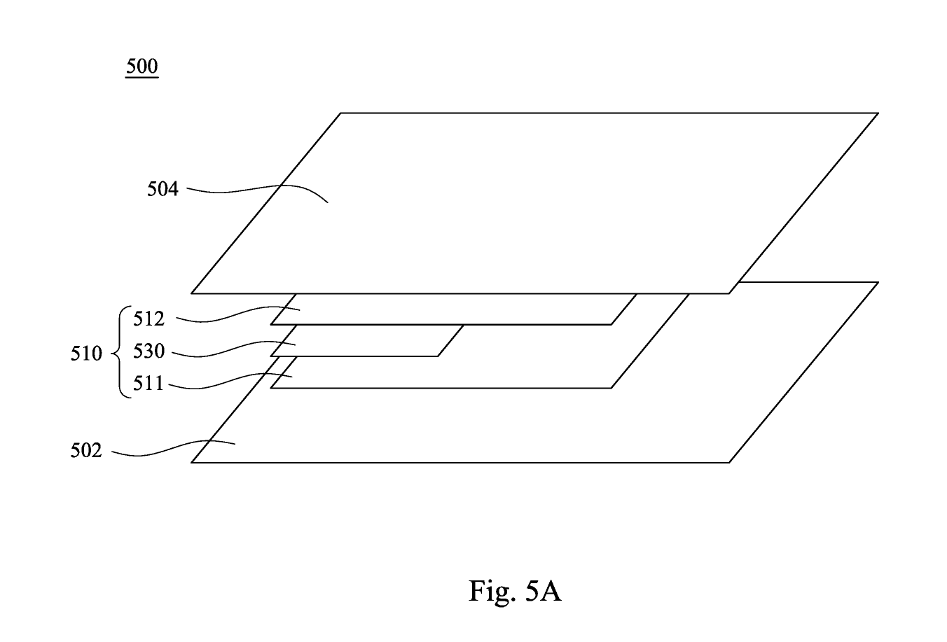

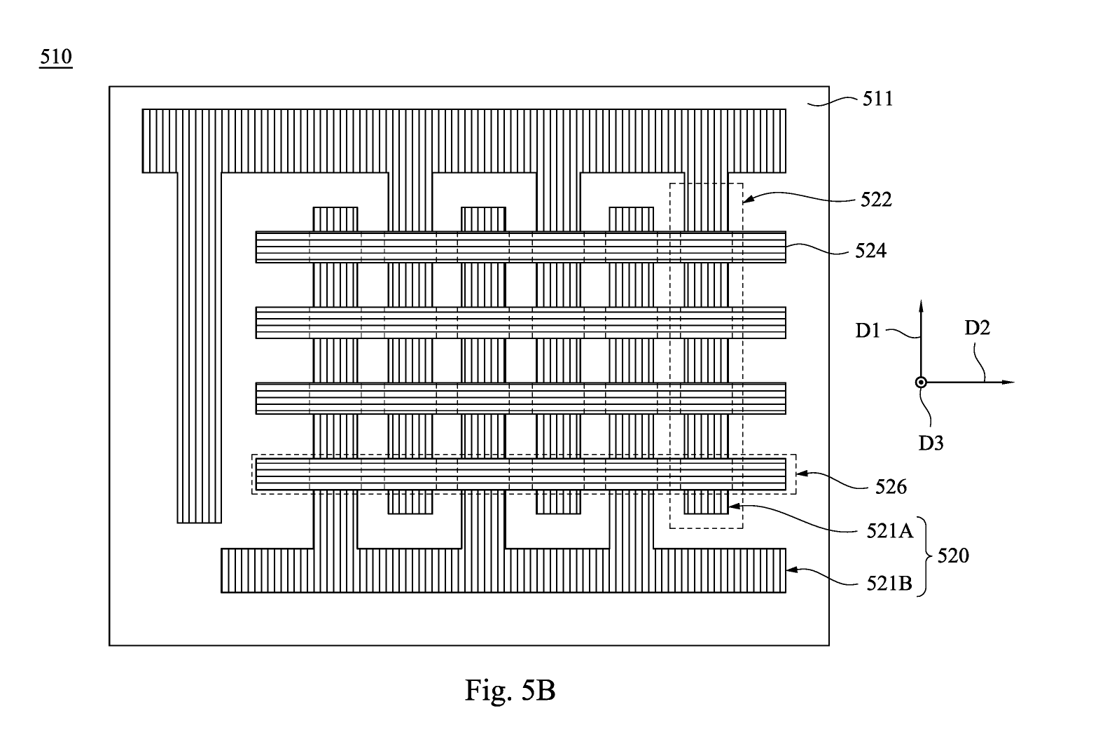

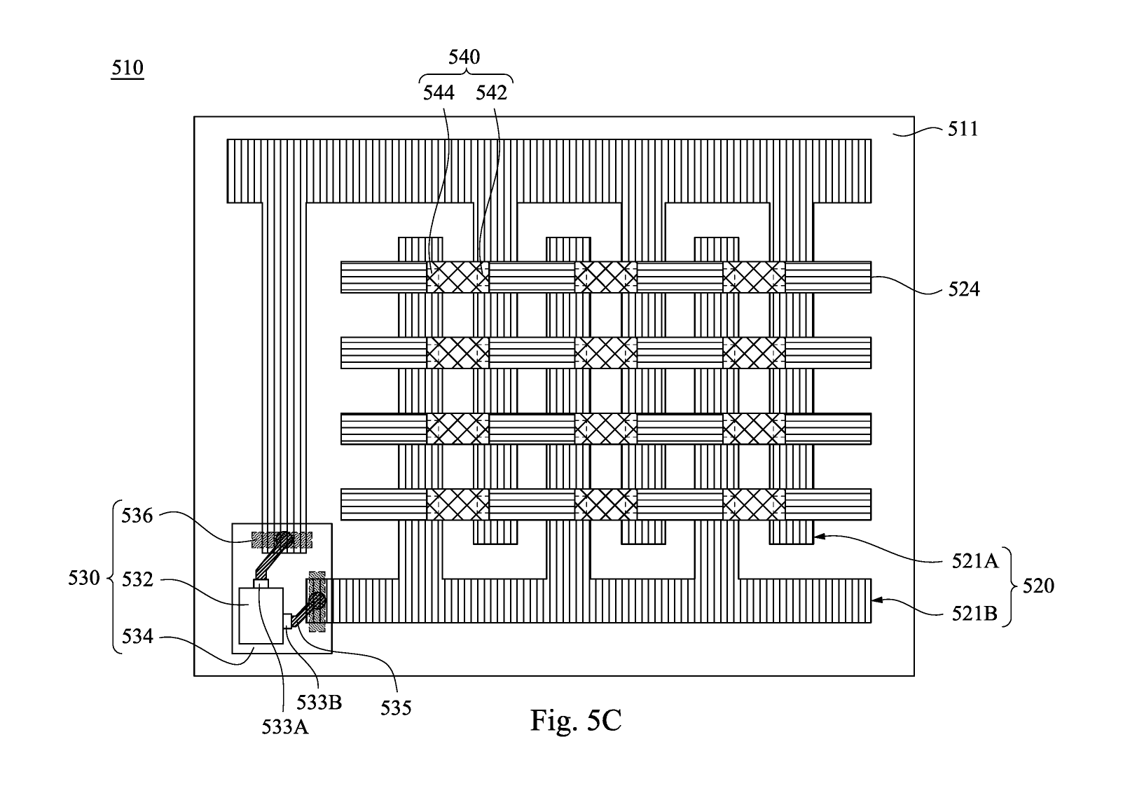

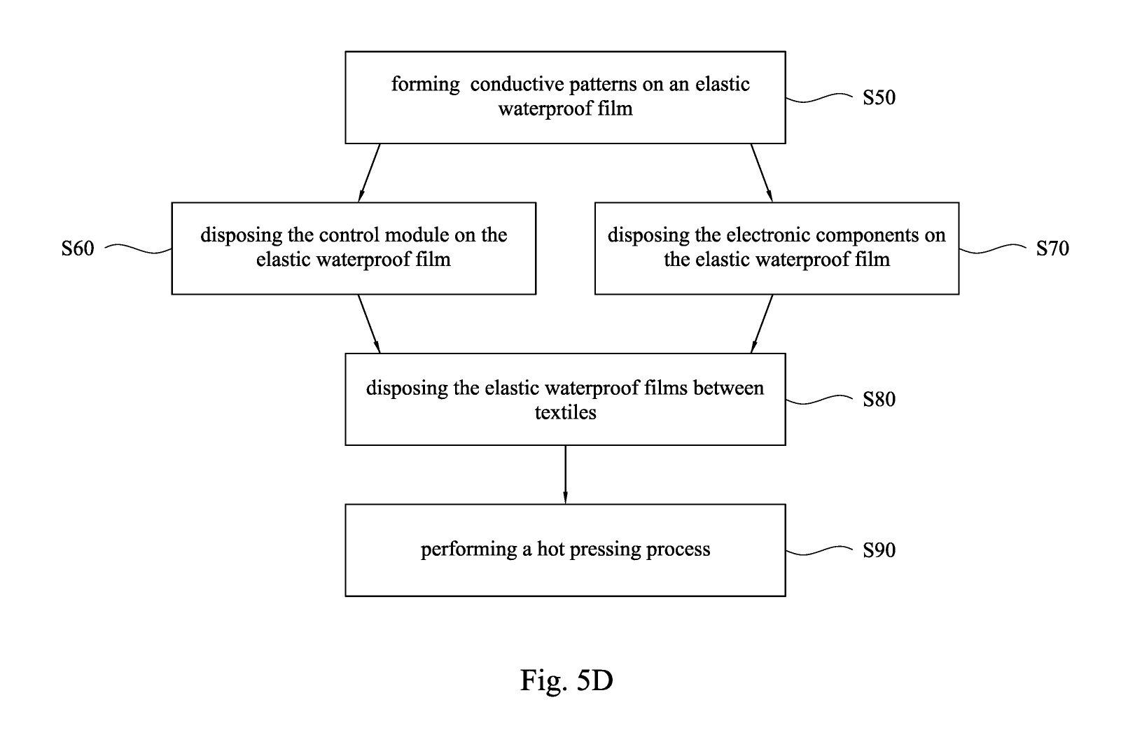

FIG. 5A is an exploded view of a fabric module 500 according to a fifth embodiment of the present disclosure. At least one difference between the smart fabric 500 of the present embodiment and the smart fabric 300 of the third embodiment is that the fabric module 510 has a light-emitting function. As shown in FIG. 5A, the fabric module 500 includes a first textile 502, a second textile 504, and a fabric module 510, in which the fabric module 510 includes a first elastic waterproof film 511, a second elastic waterproof film 512, and a control module 530. The first textile 502 is opposite to the second textile 504, and the first elastic waterproof film 511, the second elastic waterproof film 512, and the control module 530 are enclosed between the first textile 502 and the second textile 504.

The first elastic waterproof film 511 is disposed on the first textile 502, and the second elastic waterproof film 512 is disposed on the first elastic waterproof film 511. The first and second elastic waterproof films 511 and 512 may include a TPU material therein. The control module 530 is disposed between the first and second elastic waterproof films 511 and 512. In addition, the fabric module 510 includes at least one conductive pattern and at least one electronic component, such that the fabric module 510 is functional. The descriptions with respect to the functionality of the fabric module 510 are provided in the following.

FIG. 5B is a top view of the first elastic waterproof film 511 and first and second conductive patterns 520 and 524 thereon of the fabric module 510 illustrated in FIG. 5A, in which the “top view” means FIG. 5B is a view from the second elastic waterproof film 512 to the first elastic waterproof film 511 of FIG. 5A. In order to simplify FIG. 5B, the first textile 502, the second textile 504, and the second elastic waterproof film 512 are omitted in FIG. 5B. As shown in FIG. 5B, the first and second conductive patterns 520 and 524 are collectively adhered to a surface of the first elastic waterproof film 511, in which the second conductive pattern 524 is adhered to some portions of the first conductive pattern 520. That is, the first and second conductive patterns 520 and 524 may be partially overlapped with each other on the first elastic waterproof film 511.

The first conductive pattern 520 has a plurality of first row patterns 522 extending along a first direction D1. The first conductive pattern 520 can be divided into a first conductive region 521A and a second conductive region 521B. The first and second conductive region 521A and 521B are separated from each other, such that the first row patterns 522 within the first conductive region 521A are electrically isolated from the first row patterns 522 within the second conductive region 521B. In addition, the first row patterns 522 within the first conductive region 521A are electrically connected to each other, and the first row patterns 522 within the second conductive region 521B are electrically connected to each other. On the other hand, the first conductive pattern 520 can be formed by using a silver adhesive.

The second conductive pattern 524 has a plurality of second row patterns 526 electrically isolated from each other and extending along a second direction D2. The second direction D2 can intersect the first direction Dl. For example, the second direction D2 may be orthogonal to the first direction D1. Furthermore, each of the second row patterns 526 partially overlaps with the first and second conductive regions 521A and 521B of the first conductive patterns 520, and each of the overlapping regions may be rectangle. On the other hand, the second conductive pattern 524 can be formed by using at least one anisotropic conductive film, and the anisotropic conductive film is conductive in a third direction D3 which can referred to as a normal direction of FIG. 5B.

FIG. 5C is a configuration of electronic components 540 of the fabric module 510. In order to simplify FIG. 5C, the first textile 502, the second textile 504, and the second elastic waterproof film 512 are omitted in FIG. 5C. As shown in FIG. 5C, the control module 530 includes a controller 532, a flexible circuit board 534, and an anisotropic conductive film 536, in which the controller 532, the flexible circuit board 534, and the anisotropic conductive film 536 are disposed on the first and second elastic waterproof films 511 and 512.

The controller 532 includes pins 533A and 533B, and the pins 533A and 533B of the controller 532 can be electrically connected to the first conductive pattern 520 through a wire pattern 535 and the anisotropic conductive film 536. The configuration of the controller 532 regarding the pins 533A and 533B thereof which is similar to the third embodiment is not repeated herein. Furthermore, the pin 533A of the controller 532 is electrically connected to the first conductive region 521A of the first conductive pattern 520, and the pin 533B of the controller 532 is electrically connected to the first conductive region 521B of the first conductive pattern 520.

The electronic components 540 are disposed on the first elastic waterproof film 511, in which each of the electronic components 540 may be a light-emitting diode having a first pin 542 and a second pin 544. The first pins 542 and the second pins 544 are respectively located at the overlapping regions of the first and second conductive patterns 520 and 524. For example, the first pins 542 are located on the overlapping regions of the first conductive region 521A and the second conductive pattern 524, such that the electronic components 540 can be electrically connected to the first conductive region 521A of the first conductive pattern 520 through the second conductive pattern 524. Similarly, the second pins 544 are located on the overlapping regions of the second conductive region 521B and the second conductive pattern 524, such that the electronic components 540 can be electrically connected to the second conductive region 521B of the first conductive pattern 520 through the second conductive pattern 524 as well.

By the above configuration, when the pins 533A and 533B of the controller 532 respectively have different electric potentials (e.g. a positive electric potential and an negative electric potential), each of the electronic components 540 can be biased through the first and second pins 533A and 533B such that the electronic components 540 can emit light therefrom. In other words, by the above configuration, the fabric module 500 can be provided a light-emitting function.

Reference is made back to FIG. 5D. The first conductive pattern 520, the second conductive pattern 524, the control module 530, and the electronic components 540 which are enclosed between the first and second elastic waterproof films 511 and 512 may be arranged similarly to the third embodiment, and therefore the fabric module 500 is washable. FIG. 5D is a flowchart of a method for forming the fabric module 500 illustrated in FIG. 5A. As shown in FIG. 5D, the method for forming the fabric module 500 includes operations S50-S90.

The operation S50 is performed by forming conductive patterns on the elastic waterproof film. In the operation S50, the first and the second conductive patterns can be formed on the first elastic waterproof film. The first conductive pattern can be formed by applying at least one conductive ink, and the second conductive pattern can be formed by applying at least one anisotropic conductive film. For example, at least one silver adhesive can be applied to the first elastic waterproof film and then be baked to form the first conductive pattern, in which the silver adhesive can baked by a temperature of about 100° C. in about ten minutes. Then, the anisotropic conductive film can be applied to the first elastic waterproof film and some portions of the first conductive pattern, so as to form the second conductive pattern. Furthermore, as descried above, the anisotropic conductive film used for forming the second conductive pattern may be a solid anisotropic conductive tape or a liquid anisotropic conductive film.

The operation S60 is performed by disposing the control module on the elastic waterproof film. In the operation S60, the controller can be bonded to the flexible circuit board. Then, the anisotropic conductive film is arranged on the first and second conductive patterns. After arranging the anisotropic conductive film, the wire pattern of the flexible circuit board is aligned to and connected to the anisotropic conductive film, and then a hot pressing can be performed such that the flexible circuit board is further fixed on the first elastic waterproof film through the anisotropic conductive film.

The operation S70 is performed by disposing the electronic components on the elastic waterproof film. In the operation S70, the first and second pins of each of the electronic components can be aligned to and connected to the overlapping regions of the first and second conductive patterns, such that the electronic components can be electrically connected to the first conductive pattern through the second conductive pattern. Furthermore, after disposing the electronic components, a hot pressing process can be performed, so as to further fix the first and second pins of each of the electronic components on the second conductive pattern.

The operation S80 is performed by disposing the elastic waterproof films between textiles. In the operation S80, the second elastic waterproof film can be arranged to cover the first elastic waterproof film, in which the control module is covered with the second elastic waterproof film as well. Then, the first and second textiles can be used for enclosing the first and second elastic waterproof films and the control module which is disposed there between.

The operation S90 is performed by performing a hot pressing process. In the operation S90, similarly to the third embodiment, the elastic waterproof films can adhere to each other through the hot pressing process, and the first and second elastic waterproof films can respectively adhere to the first and second textiles as well. After performing the hot pressing process on the elastic waterproof films, a manufacture process for the fabric module is finished.

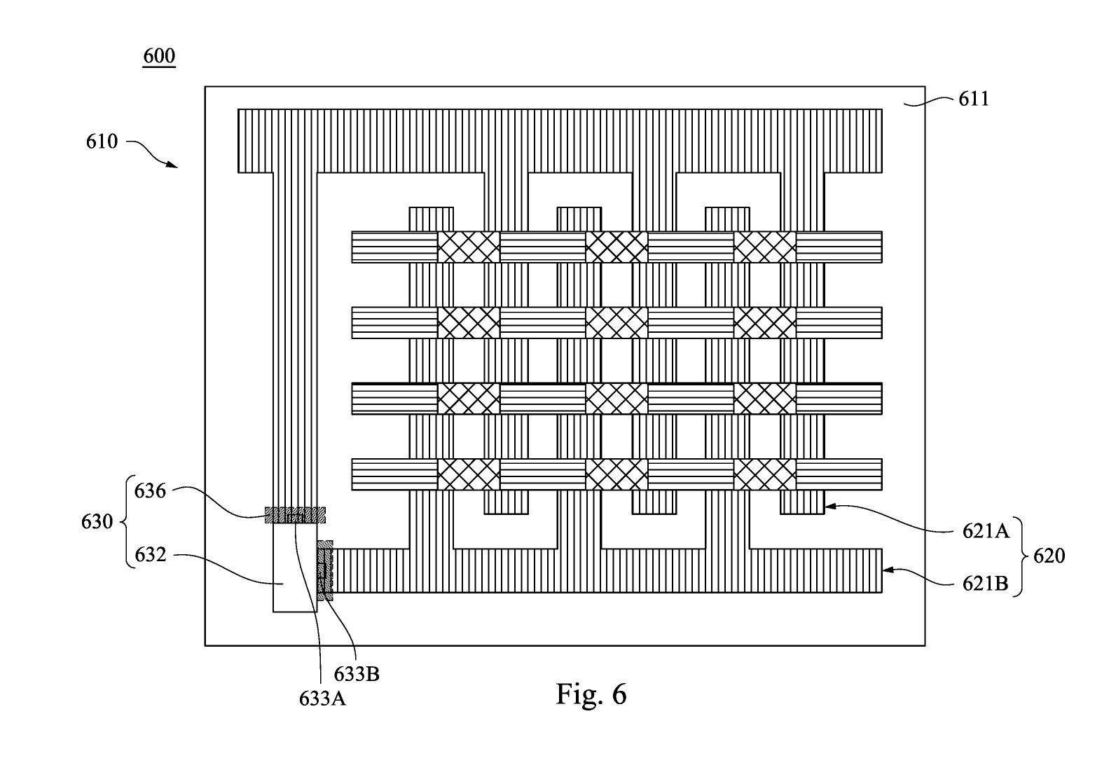

FIG. 6 is a top view of a fabric module 600 according to a sixth embodiment of the present disclosure. In order to simplify FIG. 6, the first textile, the second textile, and the second elastic waterproof film of the fabric module 600 are omitted in FIG. 6. At least one difference between the smart fabric 600 of the present embodiment and the smart fabric 500 of the fifth embodiment is that the fabric module 610 includes at least one anisotropic conductive film 636 to replace the flexible circuit board (e.g., the flexible circuit board 634), and thus the pins 633A and 633B of the controller 632 of the control module 630 are directly fixed on the first and second conductive regions 621A and 621B of the first conductive pattern 620 through the anisotropic conductive film 636. That is, the controller 632 is directly connected to the first conductive pattern 620 through the anisotropic conductive film 636.

Furthermore, at least one difference between a method for manufacturing the fabric module 600 and the method for manufacturing the fabric module 500 is that the controller 632 is directly disposed on the first elastic waterproof film 611 during the operation S60 as described in FIG. 5D, in which the pins 633A and 633B of the controller 632 are aligned to and connected to the anisotropic conductive film 636.

In aforementioned embodiments, the smart fabric includes the two textiles and the fabric module, in which the fabric module includes the more than two elastic waterproof films, the conductive pattern, and the control module. The fabric module can be provided the function through the conductive pattern and the control module, such as detecting the physiological signal, the touch function, or the light-emitting function. The conductive pattern and the control module are enclosed between the more than two elastic waterproof films, and the elastic waterproof films are disposed between the two textiles. According to such configuration, the conductive patterns and the control module can be enclosed in the space between the elastic waterproof films, so as to avoid affecting by the moisture or the dust. Accordingly, the smart fabric is washable. On the other hand, when the smart fabric is tensed, the conductive pattern thereof would not become open circuit caused from the deformation, that is, the smart fabric is stretchable.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the invention. In view of the foregoing, it is intended that the present disclosure cover modifications and variations of the present disclosure provided they fall within the scope of the following claims.

What is claimed is:

1. A fabric module, comprising:
a first textile;
a first elastic waterproof film disposed on the first textile;
a second elastic waterproof film disposed on the first elastic waterproof film;
a first conductive pattern enclosed between the first and second elastic waterproof films and adhering to a surface of the first elastic waterproof film or second elastic waterproof film;
a control module disposed on the first textile and electrically connected to the first conductive pattern; and
a second textile opposite to the first textile, wherein the first elastic waterproof film, the second elastic waterproof film, and the control module are present between the first and second textiles.

2. The fabric module of claim 1, wherein the control module comprises:
a controller disposed between the first and second elastic waterproof films; and
a flexible circuit board disposed between the first and second elastic waterproof films, wherein the controller is electrically connected to the first conductive pattern through the flexible circuit board.

3. The fabric module of claim 1, wherein the control module comprises:
a controller disposed between the first and second elastic waterproof films; and
an anisotropic conductive film, wherein the controller is electrically connected to the first conductive pattern through the anisotropic conductive film.

4. The fabric module of claim 1, wherein the first conductive pattern adheres to the surface of the first elastic waterproof film, and the fabric module further comprises:
a third elastic waterproof film disposed between the second elastic waterproof film and the second textile; and
a second conductive pattern adhering to a surface of the second elastic waterproof film and enclosed between the second and third elastic waterproof films, wherein the control module is electrically connected to the second conductive pattern.

5. The fabric module of claim 4, wherein the first conductive pattern has a plurality of first row patterns extending along a first direction, and the second conductive pattern has a plurality of second row patterns extending along a second direction which intersects the first direction.

6. The fabric module of claim 4, wherein the control module comprises:
a controller disposed between the first and third elastic waterproof films; and
a flexible circuit board disposed between the first and third elastic waterproof films, wherein the controller is electrically connected to the first and second conductive patterns through the flexible circuit board.

7. The fabric module of claim 4, wherein the control module comprises:
a controller disposed between the first and third elastic waterproof films and having a plurality of pins, wherein a vertical projection of the pins on the first elastic waterproof film partially overlaps with the first conductive pattern, and the vertical projection of the pins on the second elastic waterproof film partially overlaps with the second conductive pattern; and
an anisotropic conductive film disposed at the pins of the controller, wherein the controller is electrically connected to the first and second conductive patterns through the anisotropic conductive film.

8. The fabric module of claim 1, wherein the first conductive pattern has a plurality of first row patterns extending along a first direction, and the fabric module further comprises:
a second conductive pattern enclosed between the first and second elastic waterproof films, wherein the first and second conductive patterns collectively adhere to the surface of the first elastic waterproof film or the second elastic waterproof films, the second conductive pattern has a plurality of second row patterns extending along a second direction which intersects the first direction, and the first and second conductive patterns on the first elastic waterproof film partially overlap with each other.

9. The fabric module of claim 8, wherein the second conductive patterns are made of an anisotropic conductive film, and the anisotropic conductive film is conductive in a third direction which intersects a plane composed of the first and second directions.

10. The fabric module of claim 9, further comprising:
an electronic component enclosed between the first and second elastic waterproof films and having a first pin and a second pin, wherein the first and second pins are respectively located at overlapping regions of the first and second conductive patterns.

11. The fabric module of claim 10, wherein the first conductive pattern comprises a first conductive area and a second conductive area which are separated from each other, wherein a portion of the anisotropic conductive film is located between the first pin and the first conductive area, and another portion of the anisotropic conductive film is located between the second pin and the second conductive area.

12. The fabric module of claim 1, wherein the first and second elastic waterproof films comprise a thermoplastic urethane (TPU) material.

13. The fabric module of claim 1, wherein the first conductive pattern comprises silver particles.

14. A smart fabric, comprising:
a first textile having an inner surface and an outer surface;
a fabric module disposed at the inner surface of the first textile, wherein the fabric module comprises:
a first elastic waterproof film disposed on the first textile;
a second elastic waterproof film disposed on the first elastic waterproof film;
a first conductive pattern enclosed between the first and second elastic waterproof films and adhering to a surface of the first elastic waterproof film or second elastic waterproof film; and
a control module disposed on the first textile and electrically connected to the first conductive pattern; and
a second textile opposite to the first textile, wherein the first elastic waterproof film, the second elastic waterproof film, and the control module are present between the first and second textiles.

15. The smart fabric of claim 14, wherein the first conductive pattern comprises at least one detection electrode and a conductive path, and a thickness of each of the detection electrode and the conductive path is in a range from 10 μm to 20 μm.

16. The smart fabric of claim 14, wherein the first conductive pattern adheres to the first elastic waterproof film, the second elastic waterproof film and the second textile collectively have an opening, and the first conductive pattern is exposed from the opening.

17. The smart fabric of claim 16, wherein the fabric module is a detection module, and the first conductive pattern exposed from the opening is a detection electrode.

18. The smart fabric of claim 14, wherein the control module is enclosed between the first and second elastic waterproof films, and the control module comprises a wireless charging device and a wireless emitting-and-receiving device.
